(19) United States
(12) Patent Application Publication
(10) Pub. No.: US 2016/0145623 A1
(43) Pub. Date: May 26, 2016

(54) METHOD FOR THE TARGETED KILLING OF CELLS BY NUCLEOTIDE MOLECULES THAT ARE DIRECTED TO mRNA BINDING, AND ALSO NUCLEOTIDE MOLECULES AND APPLICATION KIT FOR SUCH USE

(71) Applicant: FRIEDRICH-SCHILLER-UNIVERSITAET, Jena (DE)

(72) Inventors: Mirko LUDWIG, Jena (DE); Tobias POEHLMANN, Zwickau (DE); Rolf GUENTHER, Hamburg (DE); Juliane REICHE, Jena (DE)

(21) Appl. No.: 14/770,877

(22) PCT Filed: Feb. 26, 2014
(86) PCT No.: PCT/EP2014/053666

§ 371 Date: Aug. 27, 2015

(30) Foreign Application Priority Data
Feb. 27, 2013 (DE) 10 2013 003 869.3

(51) Int. Cl. C12N 15/113; A61J 1/06; A61M 5/178; A61K 45/06; A61K 31/713

(52) U.S. Cl. CPC C12N 15/1137; C12N 15/113; A61K 45/06; A61K 31/713; A61M 5/178; A61J 1/06; C12N 2310/14; C12N 2320/31

(57) ABSTRACT

Nucleotide molecules are used for the targeted killing of cells, which bind with a nucleotide sequence to a single region of the mRNA which, according to sequencing analyses, is statistically very rarely subject to a mutation and, thus, also in case of increased mutation rates in the whole genome, reliably kills the cell without further mRNA binding or other influence on the cell being necessary.

### BACKGROUND OF THE INVENTION

The invention relates to a method for the targeted killing of cells by nucleotide molecules that are directed to mRNA binding as well as to exemplary nucleotide molecules and an application kit for such use.

Methods with which biological cells are to be killed in a targeted manner usually use physical means such as UV radiation, heat, etc. (Hsie A W, Brimer P A, Mitchell T J, Gosslee D G, “The dose-response relationship for ultraviolet-light-induced mutations at the hypoxanthine-guanine phosphoribosyltransferase locus in Chinese hamster ovary cells”, Somatic Cell Genet. 1975 October; 1(4):383-9; Gillespie E H, Gibbons S A, “Autoclaves and their dangers and safety in laboratories”, J Hyg (Lond). 1975 December; 75(3)475-87) or chemical substances, for example acids, bases, formaldehyde, (National Toxicology Program, “Final Report on Carcinogens Background Document for Formaldehyde” Rep Carcinog Backgr Doc. 2010 January; (10-5981):i-512) which destroy the structure of the cell per se. These means are often environmentally harmful and can hardly be applied in the organism. In order to kill cells in an organism, biochemical means (protein inhibitors, antagonists, cytostatic agents, etc.) are used. (Tanaka S, Arii S. “Current status of molecularly targeted therapy for hepatocellular carcinoma: basic science”, Int J Clin Oncol. 2010 June; 15(3):235-41. Epub 2010 May 27), which strongly affect cells in their physiology and, thus, may also lead to cell death. However, none of these methods allows targeted killing of specific cell types in the organism since said substances have the same effect on all cells.

A molecular-biological approach to influence cells in a targeted manner is the use of short double-stranded RNA. These so-called siRNA (short interfering RNA) molecules can usually interact with the mRNA of the target gene after their activation and, together with specific endoribonucleases, they form an RNA protein complex which is referred to as “RISC” (RNA induced silencing complex). The RISC complex binds to the target mRNA with endonucleases cutting the target mRNA. In this way, gene expression is avoided and the formation of target proteins is inhibited.

The inhibition of gene expression by introduction of short (19-23 bp), double-stranded RNA molecules (siRNA) in eukaryotic cells which are specific for a sequence fragment of the mRNA of a target gene was already described (Elbashir S M et al.: “Duplexes of 21-nucleotide mRNAs mediate mRNA interference in cultured mammalian cells”, Nature, 2001, May 24, 411(6836), 494-8; Liu Y et al. “Efficient and isoform-selective inhibition of cellular gene expression by peptide nucleic acids”, Biochemistry, 2004, Feb. 24, 43(7), 1921-7; U.S. Pat. No. 5,898,031 A; U.S. Pat. No. 7,056,704 B2).

The use of such molecules does not prevent the transcription of the gene and the production of an mRNA, but the siRNA initiates a cell mechanism which degrades the target mRNA. Finally, as described, the formation of a specific protein is suppressed without the expression of further genes being affected (post-transcriptional gene silencing).

Methods to transfect cells of a target tissue in vivo with siRNA to a higher degree (Ikeda et. al., “Ligand-Targeted Delivery of Therapeutic siRNA”, Pharmaceutical Research, Vol. 23, No. 8 2006, August) or to achieve a defined cell specificity by means of binding of short peptides which are cleaved in a cell-specific manner (WO 2008/098569 A2) were also developed. The use of these modified siRNA molecules allows the selective reduction or prevention of gene expression in specific cells.

If the siRNA sequence used is specific for vital genes of the cell, this may cause the death of the cell (e.g., WO 2012/098234 A1). This process can optionally also be applied cell-specifically by the mechanisms already mentioned.

In order to illustrate the known prior art, FIG. 1 provides an overview of the survival rate of breast cancer cells (MCF 7) after threefold transfection of siRNA molecules, designed according to the established and standardized BLAST methods. It is evident that the use of siRNA molecules, which are designed according to standardized BLAST methods, is not suitable for the targeted killing of cells, as the cells find ways to compensate the down-regulation of a single gene; possibly, mutations may occur in the binding site of the siRNA which makes the siRNA ineffective (escape mutation).

However, it is a disadvantage that silencing a single gene or a few genes, respectively, does not necessarily lead to the death of the cell (compensation or escape mutation as mentioned before); thus, multiple genes, which are essential for the cell, are silenced simultaneously in order to achieve the desired effect.

The prior art describes, for example, siRNAs which inhibit growth of Polo-like kinase (PLK) overexpression associated tumor cells (DE 100 11 530 A1). It also describes siRNAs which are able to inhibit the expression of human gene PLK-1 (WO 2006/035 515 A1, WO 2009/044 793 A1).

However, when using nucleotide molecules which silence multiple genes at the same time, unspecific effects are caused when used in vivo, in particular if said nucleotide molecules are to be effective cell-specifically in a particular cell type. It became clear in practice that using nucleotide molecules in order to silence multiple genes that are essential for the cell causes serious side effects.

Moreover, the problem is that mutations frequently occur in the genome of virus-infected cells or tumour cells, This is relevant in particular as when only one siRNA sequence which is specific for a target gene is used, the siRNA molecules can become ineffective due to a mutation at the site that is relevant for the siRNA and thus, the intended influence on the cell fails or can at least not he used effectively. This problem is intensified by the fact that in using nucleotide molecules, which cause cell death, a selection pressure is exerted.

In order to reliably cause cell death all the same, silencing multiple genes, which entails said side effects, or using other toxic measures, which have disadvantages and side effects also known in current practice, remains the only option.

### SUMMARY OF THE INVENTION

The problem underlying the invention is to kill cells in the organism in a broad field of use, in an effective and reliable manner and as efficiently as possible even in the case of genome mutations, without the occurrence of the aforementioned disadvantages and side effects of well-known chemical, physical, biochemical and in particular molecular-biological methods.

It was surprisingly found that nucleotide molecules which serve to influence only one single target gene of a cell and which only have homology to said target gene in a selected region, which—as was equally surprisingly found by means of sequencing analyses—statistically only very rarely have a mutation even in the case of mutations in other regions of the genome, can reliably kill the cell without further mRNA binding or other cell-influencing being necessary.

The selected regions of a nucleotide sequence which statistically rarely or very rarely are subject to mutation, even in the case of mutations in other regions of the genome, can be identified by means of sequence analyses according to the invention, In order to identify said regions, potential target regions of siRNA sequences (identified in advance) can be sequenced in corresponding cell lines, suitable for the purpose, at a given time (time t₀).

Then, the cell lines can be subjected to a selection pressure and can be cultivated (for example by adding chemotherapeutics). As a comparison, the same cell line is cultivated without selection pressure as a control. After a given time, both cell lines are analyzed regarding genome stability by means of sequence analyses. Nucleotide sequence regions, which, compared to the situation at the time t₀, do not or only slightly change in the cell line, which is subjected to selection pressure, are referred to as genetically stable and are rarely subject to mutation events. These regions, areas or sections are identified as the regions of the nucleotide sequence which statistically rarely or very rarely are subject to mutation, even in the case of mutations in other regions of the genome.

MCF-7 cells, for example, can be cultivated for 4 weeks and passaged (subcultured) on a regular basis with and without subtoxic concentrations of gemcitabine. After cell cultivation, the targeted target regions of potential siRNA sequences are sequenced once more and compared with the initial situation (time t₀). In doing so, regions can be detected which show mutations more frequently than other regions. Genetically more stable regions (meaning regions which rarely have mutation events) are to be preferred in the selection of the siRNA target regions. In concrete terms, mutations or mutation events are deletions, insertions, DNA breaks, as well as further modifications in the DNA or its structure, for example methylations. This definition further includes the fusion of chromosomes or chromosomal breakage, as well as the loss of entire chromosomes. Stable regions are those regions of the MCF-7 cells which have no mutations without selection pressure after 40 or more cell generations, while corresponding cells which were cultivated under selection pressure have no mutation(s) after 30 or more cell generations.

Thus, in connection with the present invention, regions are referred to as “stable” (or as areas, which by means of sequence analyses statistically only rarely or vary rarely are subject to a mutation) which have no mutations without selection pressure after 40 or more cell generations, while corresponding cells which were cultivated under selection pressure have no mutation(s) after 30 or more cell generations. According to the invention, said stable regions are suitable as a target for the nucleotide molecules of the invention. By contrast, areas are not considered stable (or as areas, which by means of sequence analyses statistically are more frequently subject to a mutation), when they have one or multiple mutation(s) without selection pressure after less than 40 cell generations, while corresponding cells which were cultivated under selection pressure have one or more mutation(s) after less than 30 cell generations.

In this context, the genome of the cell may even mutate or be mutated in a significant way in regions other than those selected for the binding of the nucleotide sequence and, thus, the spatial structure of the mRNA may be significantly changed; however, also in these cases, reliable cell death is induced by the method of the invention alone (and without alternative or additional treatment of the cells).

As a consequence of the suggested killing of cells by means of targeted expression inhibition of only one target gene, possible side effects of said expression are reduced to a minimum; however, the cell is reliably killed.

Among others, siRNA sequences selected for this purpose are to be disclosed for the target genes PLK, CHMP, PDCD, RFWD, ATAP and AGAP. Using siRNA sequences which are specifically designed for these genes showed that turning off the expression leads to toxic effects, without cells becoming resistant to treatment with corresponding siRNAs, even when the same siRNA sequence was used for a longer period.

This is particularly useful, as with influencing tumor cells by means of siRNA, for example, mutations may occur which make a particular siRNA ineffective so that these cells have a growth advantage and increased cell division. Surprisingly, this was not observed by using specific sequences for said genes.

Thus, the problem of the present invention is solved by a method for the targeted killing of cells by nucleotide molecules that are directed to mRNA binding, characterized in that the nucleotide molecules bind with a nucleotide sequence to a single selected region of the mRNA which, according to sequencing analyses, is statistically only very rarely subject to mutation and, thus, also in case of increased mutation rates in the whole genome, reliably kills the cell without further mRNA binding or other influence on the cell being necessary.

Moreover, according to the invention, nucleotide molecules are provided, consisting of mRNA, siRNA, miRNA, PNA, DNA, LNA or nucleotides that are completely or partially chemically modified, in particular with a size of 10-300 bp or in the case of single strands of 10-300 bases, for binding to an mRNA for the targeted killing of cells, characterized in that the nucleotide molecules have a nucleotide sequence which is complementary to a selected region of the mRNA which, according to sequencing analyses, is very rarely subject to mutation.

Not only nucleotide molecules with a size of 10-300 bp or in the case of single strands of 10-300 bases are comprised. In particular, also those nucleotide molecules are comprised which are more than 18, more than 10, more than 20, or preferred more than 21 bases in length. Preferred are those nucleotide molecules which are more than 25 bases in length. In a further preferred embodiment, the nucleotide molecules, within the meaning of the invention, are of more than 30, 40, 50 or more bases in length. According to the invention, nucleotide molecules for the targeted killing of cells are also provided, which are of from 21 to 10,000 bases in length. Most preferred are nucleotide molecules or nucleic acid molecules according to the invention with a length in the range of from 18, 19, 20, 21, 22 23, 25, 30, 40 or 50 to 100 or in the range of from 18, 19, 20, 21, 22, 23, 25, 30, 40 or 50 to 200 or in the range of from 18, 19, 20, 21, 22, 23, 25, 30, 40 or 50 to 300 bases, in particular in the range from 23 to 100 bases. Those lengths can be typically found in nucleotide molecules from the miRNA group, they are, however, not limited to said group.

As already mentioned above, the present invention provides nucleotide molecules according to the invention, consisting of mRNA, siRNA, miRNA, PNA, DNA, LNA or fully or partially chemically modified nucleotides, characterized in that the nucleotide molecules bind with a nucleotide sequence to a single selected region of the mRNA which, according to sequencing analyses, is statistically only very rarely subject to mutation and, thus, also in case of increased mutation rates in the whole genome, reliably kills the cell without further mRNA binding or other influence on the cell being necessary, wherein those nucleotide molecules are most preferred, which contain siRNA sequences, which are specific for the target genes PLK, CHMP, PDCD, RFWD, ATAP and AGAP in order to inhibit their expression. These particularly preferred nucleotide molecules of the present invention are further described hereinafter.

In a preferred embodiment, a nucleotide molecule is provided, characterized in that it contains the nucleotide sequence (5-3) UCA UAU UCG ACU UUG GUU GCC completely or partially for the purpose of an inhibition of the expression of a Polo-like kinase (PLK).

In a further preferred embodiment, a nucleotide molecule is provided, characterized in that it contains the nucleotide sequence (5-3) UCA AAC UCC AUC AUG AUC U or (5-3) UCC AUC AUG AU UUC UGG A completely or partially for the purpose of an inhibition of the expression of CHMP.

In a further preferred embodiment, a nucleotide molecule is provided, characterized in that it contains the nucleotide sequence (5-3) UUC AUA AAC ACA GUU CUC C completely or partially for the purpose of an inhibition of the expression of PDCD.

In a further preferred embodiment, a nucleotide molecule is provided, characterized in that it contains the nucleotide sequence (5-3) UCA AAU UGA GGC ACU GUG C completely or partially for the purpose of an inhibition of the expression of RFWD.

In a further preferred embodiment, a nucleotide molecule is provided, characterized in that it contains the nucleotide sequence (5-3) UUU CUU CAG AGC AGG AGC A, (5-3) AUA CAC ACC CUU UGC CUC A or (5-3) AUU UCA GGC UCA UAU UCC U completely or partially for the purpose of an inhibition of the expression of ATAP.

In a further preferred embodiment, a nucleotide molecule is provided, characterized in that it contains the nucleotide sequence (5-3) CAC AAU UCC CAC UUU GAG C, (5-3) GUU ACC CAC AAU UCC CAC U or (5-3) UUU CUU CUC UUU GUC UGG G completely or partially for the purpose of an inhibition of the expression of AGAP.

In a further preferred embodiment, a nucleotide molecule is provided, characterized in that it contains the nucleotide sequence (5-3) UAU UCU CCA AAC AAU GUG C completely or partially for the purpose of an inhibition of the expression of RCHY.

The preferred embodiments of the nucleotide molecules named for the target genes PLK, CHMP, PDCD, RFWD, ATAP and AGAP apply in the same manner for the method of the present invention disclosed above.

In a further embodiment, the present invention provides nucleotide molecules, wherein the nucleotide molecules are characterized in that they consist of mRNA, siRNA, miRNA, PNA, DNA, LNA or nucleotides that are completely or partially chemically modified, in particular with a size of 10-300 bp or in the case of single strands of 10-300 bases, for binding to an mRNA for the targeted killing of cells, characterized in that the nucleotide molecules have a nucleotide sequence which is complementary to a selected region of the mRNA which, according to sequencing analyses, is very rarely subject to mutation and, thus, also in case of increased mutation rates in the whole genome, reliably kills the cell without further mRNA binding or other influence on the cell being necessary, wherein the nucleotide molecules are selected from the group consisting of:

a nucleotide molecule, characterized in that it contains the nucleotide sequence (5-3) UCA UAU UCG ACU UUG GUU GCC completely or partially for the purpose of an inhibition of the expression of a Polo-like kinase (PLK);

a nucleotide molecule, characterized in that it contains the nucleotide sequence (5-3) UCA AAC UCC AUC AUG AUC U or (5-3) UCC AUC AUG AUC UUC UGG A completely or partially for the purpose of an inhibition of the expression of CHMP;

a nucleotide molecule, characterized in that it contains the nucleotide sequence (5-3) UUC AUA AAC ACA GUU CUC C completely or partially for the purpose of an inhibition of the expression of PDCD;

a nucleotide molecule, characterized in that it contains the nucleotide sequence (5-3) UCA AAU UGA GGC ACU GUG C completely or partially for the purpose of an inhibition of the expression of RFWD:

a nucleotide molecule, characterized in that it contains the nucleotide sequence (5-3) UUU CUU CAG AGC AGG AGC A, (5-3) AUA CAC ACC CUU UGC CUC A or (5-3) AUU UCA GGC UCA UAU UCC U completely or partially for the purpose of an inhibition of the expression of ATAP;

a nucleotide molecule, characterized in that it contains the nucleotide sequence (5-3) CAC AAU UCC CAC UUU GAG C, (5-3) GUU ACC CAC AAU UCC CAC U or (5-3) UUU CUU CUC UUU GUC UGG G completely or partially for the purpose of an inhibition of the expression of AGAP; and

a nucleotide molecule, characterized in that it contains the nucleotide sequence (5-3) UAU UCU CCA AAC AAU GUG C completely or partially for the purpose of an inhibition of the expression of RCHY.

Thus, also a method of the invention is provided for the targeted killing of cells by nucleotide molecules that are directed to mRNA binding, characterized in that the nucleotide molecules bind with a nucleotide sequence to a single selected region of the mRNA which, according to sequencing analyses, is statistically only very rarely subject to mutation and, thus, also in case of increased mutation rates in the whole genome, reliably kills the cell without further mRNA binding or other influence on the cell being necessary, wherein the nucleotide molecules are selected from the group consisting of:

a nucleotide molecule, characterized in that it contains the nucleotide sequence (5-3) UCA UAU UCG ACU UUG GUU GCC completely or partially for the purpose of an inhibition of the expression of a Polo-like kinase (PLK);

a nucleotide molecule, characterized in that it contains the nucleotide sequence (5-3) UCA AAC UCC AUC AUG AUC U or (5-3) UCC AUC AUG AUC UUC UGG A completely or partially for the purpose of an inhibition of the expression of CHMP;

a nucleotide molecule, characterized in that it contains the nucleotide sequence (5-3) UUC AUA AAC ACA GUU CUC C completely or partially for the purpose of an inhibition of the expression of PDCD;

a nucleotide molecule, characterized in that it contains the nucleotide sequence (5-3) UCA AAU UGA GGC ACU GUG C completely or partially for the purpose of an inhibition of the expression of RFWD;

a nucleotide molecule, characterized in that it contains the nucleotide sequence (5-3) UUU CUU CAG AGC AGG AGC A, (5-3) AUA CAC ACC CUU UGC CUC A or (5-3) AUU UCA GGC UCA UAU UCC U completely or partially for the purpose of an inhibition of the expression of ATAP;

a nucleotide molecule, characterized in that it contains the nucleotide sequence (5-3) CAC AAU UCC CAC UUU GAG C, (5-3) GUU ACC CAC AAU UCC CAC U or (5-3) UUU CUU CUC UUU GUC UGG G completely or partially for the purpose of an inhibition of the expression of AGAP; and

a nucleotide molecule, characterized in that it contains the nucleotide sequence (5-3) UAU UCU CCA AAC AAU GUG C completely or partially for the purpose of an inhibition of the expression of RCHY.

In a further embodiment of the above-mentioned nucleotide molecules as well as the described methods for the targeted killing of cells by nucleotide molecules that are directed to mRNA binding, the nucleotide molecules are characterized in that they are covalently or non-covalently bound to molecules such as cell-penetrating peptides and/or enzyme substrates and/or in reagents such as polyethylenimine, nanocontainers, nanoparticles or lipids and/or to receptor-ligand complexes, in particular for their introduction into cells and/or in order to achieve cell-specificity of the nucleotides.

The use of the described nucleotide molecules for the targeted killing of eukaryotic cells, in particular animal cells, as well as virus-infected, bacteria-infected or parasite-infected cells is also provided according to the invention, wherein said nucleotide molecules, in a preferred embodiment, are optionally characterized in that they are used in combination with protease inhibitors.

Said sequences can be applied in form of siRNA, shRNA, miRNA or further RNA forms as well as in the form of DNA, PNA or further nucleotide analogues in the conventional or chemically modified form.

Moreover, said sequences can be specifically introduced into target cells via delivery mechanisms as commonly known or can be activated in a targeted manner in target cells in the form of also well-known prodrug applications; both mechanisms for the induction of cell specificity can also be used in combination.

The molecules of the active ingredient can be introduced into the cells by a suitable transfection system, for example by means of nanoparticles, polyethylenimine or lipsomes in an also commonly know manner.

Moreover, the molecule constructs can be bound to further agents (for example nanoparticles as carrier system or fluorochromes) for better transportation into and to the cells, respectively, as well as for their stabilisation or for their detection.

The interferring nucleotide molecules are suitable for the targeted killing of eukaryotic cells, in particular animal, plant or fungus cells, as well as virus-infected and prokaryotic cells.

It is advantageous to use the described nucleotide molecules bound to inhibiting peptides, which are specifically cut in target cells and are thus able to activate the siRNA. In doing so, toxic effects can be generated specifically in certain cells.

When using the interferring nucleotide molecules, they can also be used in combination with protease inhibitors.

It is advantageous to have an application kit for application and administration of the interferring nucleotide molecules, consisting of at least
at least one ampoule (ampoule A) which contains the biologically effective nucleotide molecule and may further contain:
at least one further ampoule (ampoule B) with a transfection system, for example nanoparticles, polyethylenimine or lipids,
at least one further ampoule (ampoule C) which contains further components for binding the nucleotide molecules and/or for binding to a transfection system,
dilution and reaction buffers for the contents of ampoules A, B and C
one or more probes or syringes with cannula and other materials required for the injection of the mixture of the ampoule contents into the medium containing the target cells as well as
application and administration instructions.

The invention is to be further described hereinafter by means of embodiments shown in the drawings.

### BRIEF DESCRIPTION OF THE DRAWINGS

FIG. 1: Illustration of the survival rate of mammary carcinoma cells (MCF7) after threefold transfection of siRNA (conventional sequence design, without using the invention)

FIG. 2: Illustration of the survival rate of mammary carcinoma cells (MCF7) after threefold transfection of siRNA according to sequence design according to the invention.

### DETAILED DESCRIPTION OF THE INVENTION

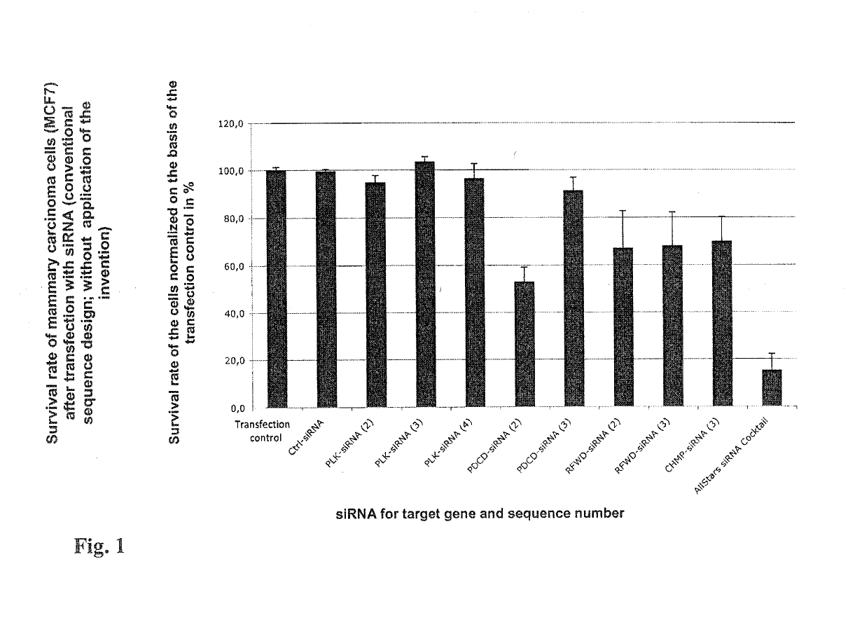

FIG. 1 shows the survival rate of breast cancer cells in an exemplary manner, which were transfected with conventionally designed siRNA sequences (without application of the invention). It is striking that the survival rate in said cells is hardly altered, even though the siRNAs are each homologous to the specific mRNA of the genes as disclosed. After threefold transfection with siRNA, the nucleotide molecules no longer influence the survival rate of the cells.

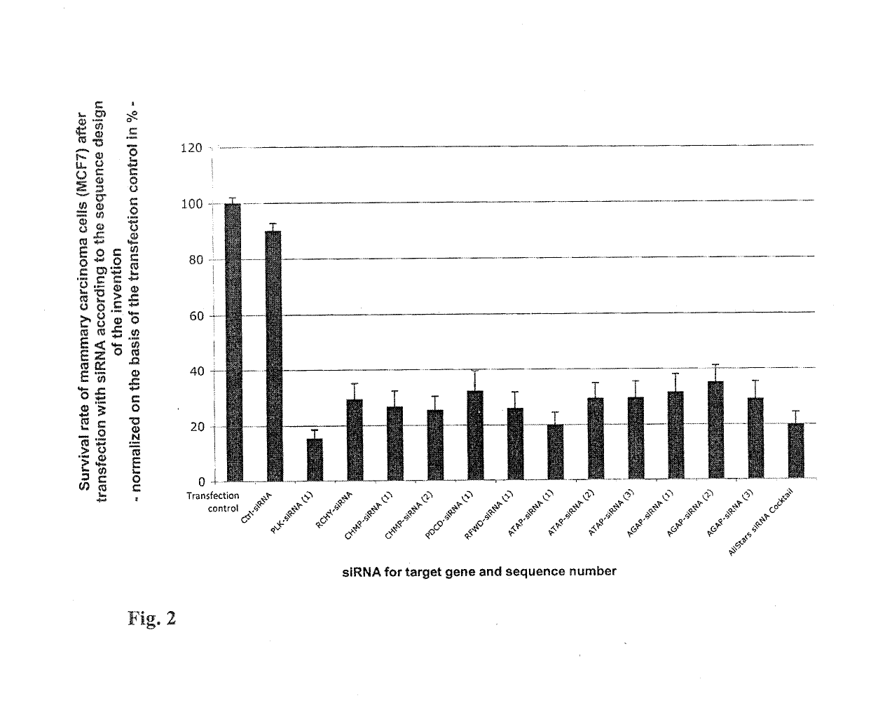

In comparison, FIG. 2 shows the survival rate of breast cancer cells which was achieved by the effect of the nucleotide molecules according to the invention. As in the embodiment of FIG. 1, the nucleotide molecules also consist of siRNA, whereas the nucleotide molecules each have a nucleotide sequence which is complementary to only one single region of the mRNA of the respective cell, wherein the region was selected according to the suggested criterion that this region—after evaluation of sequencing analyses—is statistically only vary rarely subject to mutation, irrespective of potential mutations in other regions of the cell genome.

It is evident from FIG. 2 that after threefold transfection, all nucleotide molecules based on siRNA reliably lead to a diminished survival of the cells.

Some of the siRNAs illustrated herein have an effect on the same mRNAs as in the known embodiment according to FIG. 1, they are, however, significantly more reliably effective due to the specific nucleotide sequence of the transfected molecules.

In the Figures, the following terms refer to:

transfection control: empty transfection without siRNA

Ctrl-siRNA: control transfection with a control (nonsense) siRNA

CHMP (1-3): siRNA against CHMP

PDCD (1-3): siRNA against PDCD

RFWD (1-3): siRNA against RFWD

ATAP (1-3): siRNA against ATAP

AGAP (1-3): siRNA against AGAP

PLK (1-3): siRNA against PLK

RCHY: siRNA against RCHY

Allstars siRNA cocktail: mixture of several siRNAs for induction of toxicity.

For detection, the cells to be treated were cultivated in 98 well cell culture plates and three consecutive siRNA transfections were carried out. Subsequently, the toxicity was determined by means of an XTT assay.

The introduction of toxic siRNAs into the cells is to induce cell death. The control siRNA is the negative control. Allstars is a cocktail with different toxic siRNA sequences and represents the positive control.

When the individual siRNA sequences were used, as shown in FIG. 2, it was possible to effectively achieve cell death in each case (and in a comparable manner as with the Allstars siRNA cocktail). Using the Allstars siRNA cocktail prevents the occurrence of escape mutations. With the sequences that were used specifically for this case, cell behaviour was analoguous to that of the positive control. In each case, it was possible to cause cell death reliably and effectively by silencing only one single gene.

All publications, patents and patent applications as well as other documents which are cited herein are hereby incorporated by reference.

1. A method for the targeted killing of cells, comprising binding a nucleotide sequence of nucleotide molecules to a single selected region of mRNA of the cells, the selected region being a region which, according to sequencing analyses, is statistically only very rarely subject to mutation, whereby the cells are reliably killed without further mRNA binding or other influence on the cell being necessary and without regard to the mutation rate of the whole genome of the cells.

2. Nucleotide molecules comprised of mRNA, siRNA, miRNA, PNA, DNA, LNA or nucleotides that are completely or partially chemically modified with a size of 10-300 bp or, in the case of single strands, of 10-300 bases, for binding to an mRNA for the targeted killing of cells having the mRNA, wherein the nucleotide molecules have a nucleotide sequence which is complementary to a selected region of the mRNA which, according to sequencing analyses, is very rarely subject to mutation.

3. The nucleotide molecules according to claim 2, wherein the complementary nucleotide sequence is (5-3) CAC AAU UCC CAC UUU GAG C, (5-3) GUU ACC CAC AAU UCC CAC U or (5-3) UUU CUU CUC UUU GUC UGG G, which inhibits expression of AGAP.

4. The nucleotide molecules according to claim 2, wherein the complementary nucleotide sequence is (5-3) UCA AAC UCC AUC AUG AUC U or (5-3) UCC AUC AUG AUC UUC UGG A, which inhibits expression of CHMP.

5. The nucleotide molecules according to claim 2, wherein the complementary nucleotide sequence is (5-3) UUC AUA AAC ACA GUU CUC C, which inhibits expression of PDCD.

6. The nucleotide molecules according to claim 2, wherein the complementary nucleotide sequence is (5-3) UCA AAU UGA GGC ACU GUG C, which inhibits expression of RFWD.

7. The nucleotide molecules according to claim 2, wherein the complementary nucleotide sequence is (5-3) UUU CUU CAG AGC AGG AGC A, (5-3) AUA CAC ACC CUU UGC CUC A or (5-3) AUU UCA GGC UCA UAU UCC U, which inhibits expression of ATAP.

8. The nucleotide molecules according to claim 2, wherein the complementary nucleotide sequence is (5-3) UAU UCU CCA AAC AAU GUG C, which inhibits expression of RCHY.

9. The nucleotide molecules according to claim 2, wherein the nucleotide molecules are covalently or non-covalently bound to cell-penetrating peptides or enzyme substrates or receptor-ligand complexes or are in reagents selected from the group consisting of polyethylenimine, nanocontainers, nanoparticles and lipids for their introduction into cells or in order to achieve cell-specificity of the nucleotides.

10. The method of claim 13, wherein the cells are selected from the group consisting of animal cells, and virus-infected, bacteria-infected and parasite-infected cells.

11. The method of claim 13, further comprising also treating the cells with protease inhibitors.

12. Application kit for application and administration of the nucleotide molecules according to claim 2, comprising
at least one ampoule (ampoule A) which contains the nucleotide molecules;
at least one further ampoule (ampoule B) with a transfection system comprising at least one of cell-penetrating peptides, nanoparticles, polyethylenimine or lipids;
at least one further ampoule (ampoule C) which contains further components for binding to the nucleotide molecules or for binding to a transfection system;
dilution and reaction buffers for the contents of ampoules A and B;
one or more probes or syringes with cannula and other materials required for the injection of the mixture of the ampoule contents into a medium containing the target cells; and
application and administration instructions.

13. A method for the targeted killing of cells, comprising selecting a single region of mRNA of the cells which, according to sequencing analyses, is very rarely subject to mutation and binding to the selected region the complementary nucleotide sequence of the nucleotide molecules of claim 2.


SEQUENCE LISTING (12 sequences)

SEQ ID NO: 1 - RNA, 21 nt, Artificial Sequence
  Description of Artificial Sequence Synthetic oligonucleotide
ucauauucgacuuugguugcc

SEQ ID NO: 2 - RNA, 19 nt, Artificial Sequence
  Description of Artificial Sequence Synthetic oligonucleotide
ucaaacuccaucaugaucu

SEQ ID NO: 3 - RNA, 19 nt, Artificial Sequence
  Description of Artificial Sequence Synthetic oligonucleotide
uccaucaugaucuucugga

SEQ ID NO: 4 - RNA, 19 nt, Artificial Sequence
  Description of Artificial Sequence Synthetic oligonucleotide
uucauaaacacaguucucc

SEQ ID NO: 5 - RNA, 19 nt, Artificial Sequence
  Description of Artificial Sequence Synthetic oligonucleotide
ucaaauugaggcacugugc

SEQ ID NO: 6 - RNA, 19 nt, Artificial Sequence
  Description of Artificial Sequence Synthetic oligonucleotide
uuucuucagagcaggagca

SEQ ID NO: 7 - RNA, 19 nt, Artificial Sequence
  Description of Artificial Sequence Synthetic oligonucleotide
auacacacccuuugccuca

SEQ ID NO: 8 - RNA, 19 nt, Artificial Sequence
  Description of Artificial Sequence Synthetic oligonucleotide
auuucaggcucauauuccu

SEQ ID NO: 9 - RNA, 19 nt, Artificial Sequence
  Description of Artificial Sequence Synthetic oligonucleotide
cacaauucccacuuugagc

SEQ ID NO: 10 - RNA, 19 nt, Artificial Sequence
  Description of Artificial Sequence Synthetic oligonucleotide
guuacccacaauucccacu

SEQ ID NO: 11 - RNA, 19 nt, Artificial Sequence
  Description of Artificial Sequence Synthetic oligonucleotide
uuucuucucuuugucuggg

SEQ ID NO: 12 - RNA, 19 nt, Artificial Sequence
  Description of Artificial Sequence Synthetic oligonucleotide
uauucuccaaacaaugugc